(19) United States
(12) Patent Application Publication
(10) Pub. No.: US 2019/0151003 A1
(43) Pub. Date: May 23, 2019

(54) BONE IMPLANT HAVING AN OFFSET IN THE INJECTION CHANNEL AND IMPLANTATION TOOL THEREFOR

(71) Applicant: Aesculap AG, Tuttlingen (DE)

(72) Inventors: JANINA ACKERMANN, Tuttlingen (DE); IRENE MARX, Trossingen (DE); STEPHAN LINDNER, Wurmlingen (DE); SVEN KRÜGER, Trossingen (DE); BARBARA GNOTH, Immendingen (DE)

(21) Appl. No.: 16/091,750

(22) PCT Filed: Apr. 4, 2017
(86) PCT No.: PCT/EP2017/057966

§ 371 Date: Oct. 5, 2018

(30) Foreign Application Priority Data
Apr. 6, 2016 (DE) 10 2016 106 308.8

(51) Int. Cl. A61B 17/88; A61B 17/86

(52) U.S. Cl. CPC A61B 17/8841; A61B 17/8805; A61B 17/7001; A61B 17/864; A61B 17/8625

(57) ABSTRACT

A bone implant includes a shank for arrangement in a bone and a head arranged proximally on the shank and having a tool receiver for an implantation tool. The shank has a longitudinal axis. A continuous injection channel for injection of bone connection material is formed in the shank and has a proximal inlet opening for bone connection material on the side of the tool receiver. The wall of the shank surrounding the injection channel has a radial offset with respect to the longitudinal axis on the side of the proximal inlet opening. An implantation instrument for a bone implant includes an injection cannula having an inner channel having a longitudinal axis for injection of bone connection material. The inner channel has a distal outlet opening for bone connection material. A wall of the injection cannula surrounding the inner channel has a radial offset with respect to the longitudinal axis.

### RELATED APPLICATIONS

This application is the United States national phase entry of International Application No. PCT/EP2017/057966, filed Apr. 4, 2017, which is related to and claims the benefit of priority of German Application No. 10 2016 106 308.8, filed Apr. 6, 2016. The contents of International Application No. PCT/EP2017/057966 and German Application No. 10 2016 106 308.8 are incorporated by reference herein in their entireties.

### FIELD

The present invention relates to a bone implant comprising an implant shank for arrangement in a bone and an implant head arranged proximally on the implant shank and having a tool receiver for an implantation tool, wherein the implant shank has a longitudinal axis A and a continuous injection channel for injection of a bone connection material is formed in the implant shank and has a proximal inlet opening for the bone connection material on the side of the tool receiver. The invention further relates to an implantation instrument for a bone implant comprising an injection cannula having an inner channel with a longitudinal axis B for injection of bone connection material. Finally, it relates to an implantation system comprising a bone implant according to the invention as well as an implantation instrument according to the invention.

### BACKGROUND

Bone implants such as joint prostheses, bone or pedicle screws are known from prior art. They are inserted into a bone and fastened in place therein. Their fastening can be accomplished for instance by screwing and alternatively or in addition by means of a bone connection compound or bone connection material, also referred to as bone cement. To this end, a bone implant comprises at least one channel, also named injection channel or cement channel, through which the bone connection compound or bone connection material can be introduced in the desired way and placed at the desired location during an implantation.

In the course of cementing bone implants, there is the problem that during removal of a cannula used for introducing bone cement into a dedicated channel of the implant, possibly hardened bone cement may emerge, similar to a glue thread which does not easily come off from the adhesive bottle after completion of the gluing process. Such cement components may restrict or even prevent the intended use and/or manipulation/arrangement of the implant in a detrimental way, or they may come off and present a danger for the patient. This detrimental emerging of bone cement may take place both on the implant and the cannula.

Pedicle screws serve e.g. for the dorsal stabilization of the vertebral column by means of a transpedicular screw connection. Here, pedicle screws are placed in the pedicles of respectively neighboring vertebrae, whereupon an angularly stable connection is created between the pedicle screws axially arranged one above the other and an axially extending longitudinal beam or web. In such arrangement, the longitudinal beam is inserted into a screw-head side receiving sleeve, the so-called tulip, and is clamped in place therein. In this way, the pedicle screws and the longitudinal beam form a vertebral stabilization system. In the event of a cementation of windowed pedicle screws, it may happen upon removing an augmentation cannula that a part of the hardened cement is pulled out of the pedicle screw and/or the augmentation cannula and breaks in such a way that a cement column protrudes from the pedicle screw. This may impede further operation steps or even make them impossible. Any unnoticed detachment of parts or portions of such a cement column represents a significant risk for the well-being, the health or the life of the patient.

DE 10 2013 109 895 A1 discloses an implant, e.g. in the form of a polyaxial screw having a screw shank and a longitudinal channel formed therein, and a medical instrument for implanting it, wherein the instrument comprises a tool portion having a first tool element for engagement with a corresponding second tool element in the implant, wherein a bone cement container is formed or arranged on or in the instrument. A distal end of the screw shank is open and is in fluid connection with the longitudinal channel. The proximal end of the screw shank is realized in the form of a spherical head on which a sleeve is mounted in an articulated manner. The proximal end of the longitudinal channel opens within the ball head. The instrument and the implant are to be engaged with each other by means of the two tool elements, creating a fluid connection between the bone cement container and the longitudinal channel.

DE 10 2008 024 440 A1 discloses a similar implant which comprises a tool receiver to be connected to an implantation tool and a connecting device for connecting the implant to an injection cannula for injection of a bone connection material, wherein the connecting device comprises a female thread section formed in or on the tool receiver.

DE 20 2012 008 715 U1 discloses a pedicle screw which is cannulated and provided with lateral bores or milled recesses and allows a full or partial closure of the cannulation at the distal end of the screw.

CN 2032 52718 U discloses a pedicle screw which is provided with an inner channel and through-openings in the screw shank and the thread section. A female thread for connecting the screw to an injection cannula for applying bone cement is formed in a head of the pedicle screw.

The previously described prior art only addresses the tightness between the cement cannula and the bone screw. For this purpose, threads in the cement cannula as well as in the bone screw are known. The area where the inner channel of the cement cannula and the injection channel of the bone screw come into contact with the bone cement is formed to be smooth and without undercut in known systems. Thus, it may disadvantageously happen that possibly hardened cement components are pulled out of the instrument and/or the longitudinal channel of the implant when the cement injector is detached from the implant.

### SUMMARY

Starting from the previously mentioned prior art, the invention is based on the object to provide a bone implant and a bone implantation system which do not exhibit the above disadvantages. Specifically, any emerging or even detachment of bone connection material from the implant and the medical instrument used for application is to be reliably avoided. The bone implant and the bone implantation system should have an easy and safe handling and be cost-effective.

This object is achieved according to the present invention by a bone implant wherein the wall of the implant shank surrounding the injection channel has a radial offset with respect to the longitudinal axis on the side of the proximal inlet opening. It is further achieved by an implantation instrument for a bone implant of the invention comprising an injection cannula with an inner channel having a longitudinal axis B for injection of bone connection material, wherein the inner channel comprises a distal outlet opening for bone connection material, wherein the wall of the injection cannula surrounding the inner channel has a radial offset with respect to the longitudinal axis B on the side of the distal outlet opening. Finally, it is achieved by an implantation system comprising a bone implant according to the invention and an implantation instrument according to the invention.

In other words, a bone implant is proposed which comprises a distal implant shank designed to be embedded in a bone, and a proximal implant head having a tool receiver for an implantation tool, from which an injection channel for injection of a bone connection material extends in the implant shank and has a predetermined injection channel diameter at this place, wherein the wall surrounding the injection channel starting from the tool receiver has an offset, preferably a thread machined in the wall of the injection channel or a groove machined in the wall of the injection channel, which with respect to the predetermined injection channel diameter proximally and distally relative to the offset extends radially outwards. In addition, an implantation instrument for a bone implant according to the invention is proposed, comprising an injection cannula having an inner channel with a longitudinal axis B for injection of bone connection material, wherein the inner channel comprises a distal outlet opening for bone connection material and has a predetermined inner channel diameter at this place, wherein the wall of the injection cannula surrounding the inner channel starting from the distal outlet opening has a radial offset with respect to the longitudinal axis B, preferably a thread machined in the wall of the inner channel or a groove machined in the wall of the inner channel, which with respect to the predetermined inner channel diameter distally and proximally relative to the offset extends radially outwards. Further, an implantation system comprising a bone implant according to the invention and an implantation instrument according to the invention is proposed.

Stated in functional terms, the invention discloses a static friction increasing element at a proximal end of a bone implant and at the distal end of an implantation tool, respectively, which counteracts—for instance by means of the above-mentioned offset and/or by means of increasing the roughness—a proximal movement of the bone connection material caused by the surgeon when removing the implantation tool, and hence ensures a predetermined breaking point for the bone connection material prior to/during its process of fully hardening. As will become clear in the further course of this application, the static friction increasing element may take several forms, such as a step, a groove, a thread and so on.

The bone implant according to the invention is in particular provided and suitable to be arranged with the implant shank in a bone cavity and cemented in place therein. The injection channel is formed in the implant shank so as to be continuous at least in sections. It forms a fluidic connection between the proximal inlet opening and at least one outlet opening for the bone connection material formed in the area of the implant shank. According to the concept underlying the invention, the radial offset forms in the injection cannula a retaining structure for bone connection material located in the injection channel. As an alternative or in addition, the offset may be formed and dimensioned such that a predetermined breaking point is created in the bone connection material present in the injection channel, wherein the portion in which the bone connection material hardens and the tool receiver are designed as separate portions. The tool receiver which is proximally arranged and the undercut or radial offset situated at a more distal point, fulfill different functions.

If an injection instrument is uncoupled and removed from the implanted implant after completion of the introduction of bone connection material, the connection material present in the injection channel is retained by the offset, at least in proximal direction, cannot perform a relative movement in proximal direction in the channel and cannot emerge from the channel and the implant. The same effect, but in the opposite direction, is brought about with regard to the inner channel of the injection cannula of the implantation system according to the invention: Upon removing the instrument from the implanted implant when the introduction of bone connection material is completed, the connection material present in the inner channel of the injection cannula is retained by its offset, at least in distal direction, cannot perform a relative movement in distal direction in the inner channel of the injection cannula, and cannot emerge therefrom. As a whole, the invention safely prevents that bone cement, in particular partially or fully hardened bone cement, emerges from the implant and the medical instrument used for implanting it.

As described above, the radial offset in the injection channel wall of the implant and the radial offset in the inner channel wall of the instrument serve to increase the adherence of the bone connection medium in the axial direction. For fulfilling this function, an increased roughness of the channel walls is conceivable as well.

A particular advantage of the invention is that the radial offset which according to the invention is formed in the injection channel as well as in the inner channel of the injection cannula, allows to form a defined predetermined breaking point for bone cement present therein. Thus, it is always ensured that the respectively desired amount of bone connection material remains in the implant. Such a predetermined breaking point also prevents separate particles from breaking out or partially hardened bone cement from crumbling, and also avoids a contamination of the implantation surroundings by such particles. One aim of the invention is that the cement, owing to the offset in the bone screw and the cement cannula, can break in the area between the two offsets and also does break there in a defined manner.

Advantageous embodiments of the invention will be explained in more detail below. In the description of the invention, the term “channel” concerns both the injection channel of the implant and the inner channel of the injection cannula.

One embodiment of the invention is characterized in that the radial offset is formed by steps, shoulders or any cross-sectional changes machined in the wall of the injection channel. With regard to a medical implantation instrument according to the invention, the radial offset is formed by steps, shoulders or cross-sectional changes machined in the wall of the inner channel. The steps, shoulders or cross-sectional changes form an abutment structure for the bone connection material within the channel, which structure is substantially transverse to or considerably inclined relative to the longitudinal axis A or B. Thus, it constitutes a sort of obstacle counteracting a movement of bone cement, in particular in the hardened and partially hardened state, in the direction of the respective longitudinal axis A or B, while allowing the injection of not yet hardened bone connection material without substantial limitations.

As an alternative or in addition, the offset in the implant as well as in the instrument may be formed in circumferential direction so as to circumferentially extend at least in sections, i.e. with the implant with respect to the longitudinal axis A and with the instrument with respect to the longitudinal axis B. Due to a corresponding design of the offset in circumferential direction, for instance by sharp-edged disruptions of the offset, a notch effect can be provoked in the bone connection material which favors a failure of the material at the desired predetermined breaking point. A particularly stable retaining of the cement in the respective channels is promoted by a full and continuous circumferential offset.

One embodiment of the invention is characterized in that the radial offset is formed by a thread machined in the wall of the injection channel or of the inner channel, or by an undercut machined in the wall of the injection channel or of the inner channel. Despite of the problematic accessibility and the common dimensioning of the implants concerned, such structures can be manufactured in an easy, inexpensive and cost-effective manner.

A further embodiment of the invention is characterized in that the radial offset is formed in an area of the wall which (regarding the implant) is situated near the proximal inlet opening or on the side of the proximal inlet opening, or (regarding the injection cannula) near the distal outlet opening or on the side of the distal outlet opening. It could also be said that the radial offset is formed in a portion of the channel wall which adjoins the proximal inlet opening or the distal outlet opening. The radial offset may be spaced from the proximal inlet opening or distal outlet opening in particular by at least about 0.5 mm and at most about 10 mm, with any values therebetween being possible. According to one embodiment, it may be spaced from the proximal inlet opening or distal outlet opening by a distance of between about 1 mm to about 7.5 mm, according to another embodiment between about 1.5 mm to about 5 mm and according to a further embodiment of between about 2 mm to about 3 mm.

A further embodiment of the invention is characterized in that the offset of the injection channel in radial direction with respect to the longitudinal axis A and the offset of the inner channel with respect to the longitudinal axis B amounts to between about 0.1 mm to 3 mm, preferably between about 0.2 mm to 2 mm and particularly preferred between about 0.3 mm to about 1.5 mm. In these areas, there is the advantage that not yet hardened bone connection material can still be injected through the respective channel without limitation or at least under minor limitations only, but partially or fully hardened bone connection material is safely prevented from performing movements in axial direction and is retained.

According to an embodiment of the invention, provision is made that the bone implant is a bone screw, in particular a pedicle screw. Said pedicle screw may have an implant head formed in particular as a ball head, on which a tulip for receiving and clamping a fixation rod is arranged or can be arranged in a positionable manner. Furthermore, it may comprise a tool receiver for a screw-driving tool, preferably within the ball head, wherein the proximal inlet opening is formed in a distal boundary surface of the tool receiver.

It is particularly advantageous if the tool receiver of the bone implant comprises a connection structure intended for being detachably connected to an injection cannula for injection of bone connection material. Similarly, the injection cannula may have a connection structure for a detachable connection with a tool receiver of the bone implant, in particular in the form of a thread or a bayonet structure. In this way, the instrument can be safely arranged and fixed on the implant in such a manner that any emergence of bone connection material through the joint between the implant and the instrument can be reliably avoided. The connection structure may have a design in particular in the form of a thread or a bayonet structure. These are easy to manufacture and offer a user-friendly handling. As an alternative or in addition, the tool receiver may have an engagement contour for an implantation tool, by means of which the bone implant is implanted during an implantation, in particular in the form of a hexagon socket or a Torx socket. The injection cannula may be formed as an implantation tool and may have a tool contour matching with the tool receiver, in particular in the form of a hexagon head or a Torx head.

In summary, it can be stated that the invention allows to reliably avoid the formation of cement webs upon removing a cement cannula from the implant or bone screw. A predetermined breaking point can be generated in the cement located in the channel. Said breaking point is located preferably underneath the cement cannula and at most 10 mm underneath the ball head of the bone screw. The respective undercuts are realized here such that a shear plane is formed which is equal to or larger than the smallest cross-sectional area of the respective channel of the bone screw and of the cement cannula as well.

### BRIEF DESCRIPTION OF THE DRAWING FIGURES

Further features and advantages of the present invention will be apparent from the following exemplary and non-limiting description of the invention on the base of a pedicle screw system as an example for a bone screw system with the aid of Figures. These Figures are of schematic nature only and merely serve the understanding of the invention. In the Figures:

FIG. 1 shows in a sectional view along the longitudinal axes A and B portions of an implant according to a first embodiment of the invention in the form of a pedicle screw comprising an implantation instrument according to a first embodiment of the invention in operative engagement,

FIG. 2 shows an embodiment similar to FIG. 1 in a perspective view,

FIG. 3 shows in a sectional view along the longitudinal axes A and B portions of an implant according to a second embodiment of the invention in the form of a pedicle screw comprising an implantation instrument according to a second embodiment of the invention in operative engagement,

FIG. 4 shows an embodiment of FIG. 3 in a perspective view, and

FIG. 5 shows the embodiment of FIGS. 1 and 2 with an implantation instrument that is not illustrated in cross-section.

### DETAILED DESCRIPTION

The present description of the invention on the base of the Figures is made with reference to a pedicle screw 1. However, the invention relates in particular to a bone screw. The term “pedicle screw” is to be understood as being directed to a bone screw, and vice versa.

The pedicle screw 1 has a longitudinal axis A and comprises a screw shank 2 extending in its axial direction and including a male thread 3 or bone thread 3. Formed on the proximal end of the screw shank 2 is a ball-shaped screw head 4 which for its part carries a receiving sleeve 5, also referred to as a tulip 5, so as to be able to be positioned by swiveling due to its ball shape. The tulip 5 has an essentially U-shaped design comprising opposing wall portions 6, 7, which are generally referred to as sleeve flanks, and an intermediate gap 8 extending in the radial direction and defining a receiving space for a longitudinal beam or web which is not shown in the Figures. The tulip 5 is provided with a female thread 9 extending in axial direction, into which a (not shown) clamping screw (sets crew) can be screwed after insertion of the longitudinal beam in the tulip 5, in order to clamp and fix it.

Formed in the screw shank 2 of the pedicle screw 1 is an injection channel 10 which is oriented in the direction of the longitudinal axis A and (not identifiable in the Figures) extends substantially along the entire length of the screw shank 2. The injection channel 10 comprises a proximal inlet opening 11 at the head-side end and one or more outlet openings at the distal end of the shank 2 and/or distributed over the screw shank 2. By means of the implantation instrument 12 illustrated in part in the Figures bone connection material or bone cement is conveyed through the injection channel 10 to the outlet openings of the pedicle screw 1 which is placed/screwed into a bone, which material emerges from these and cements the pedicle screw 1 in place in the bone.

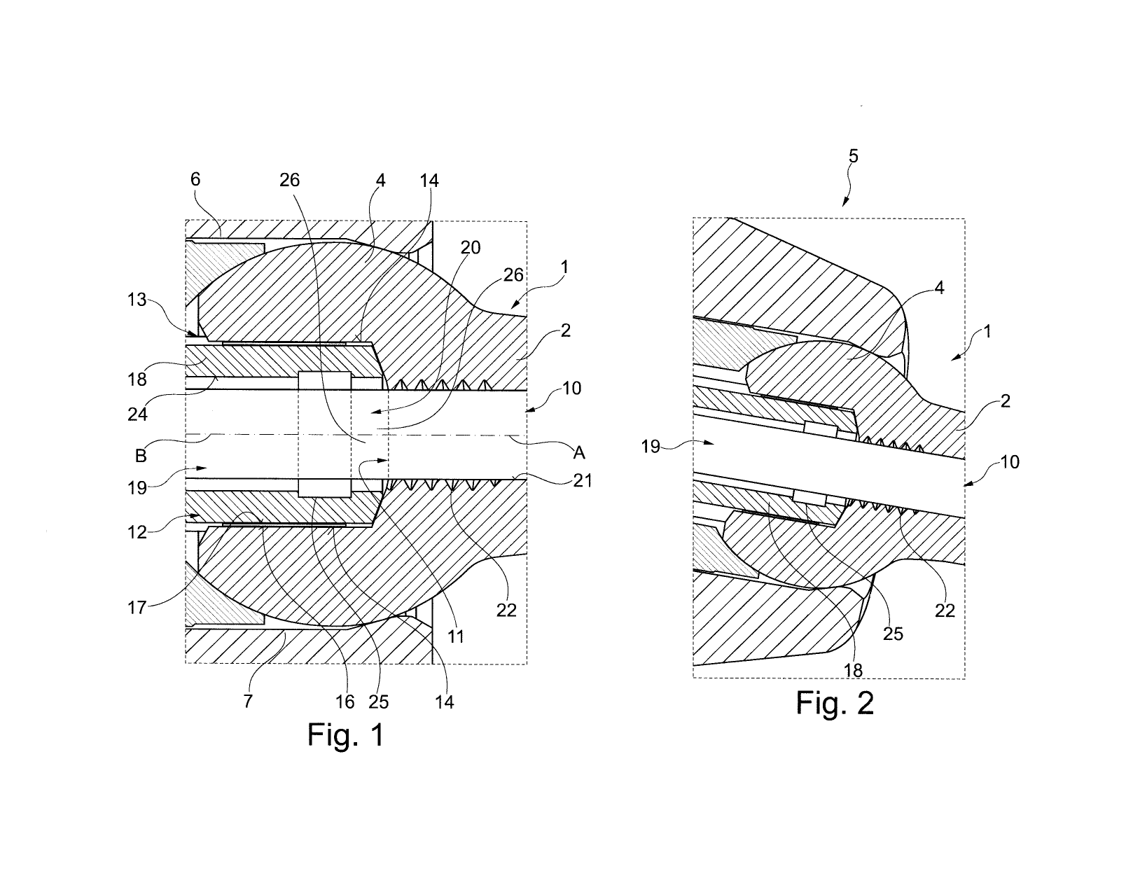

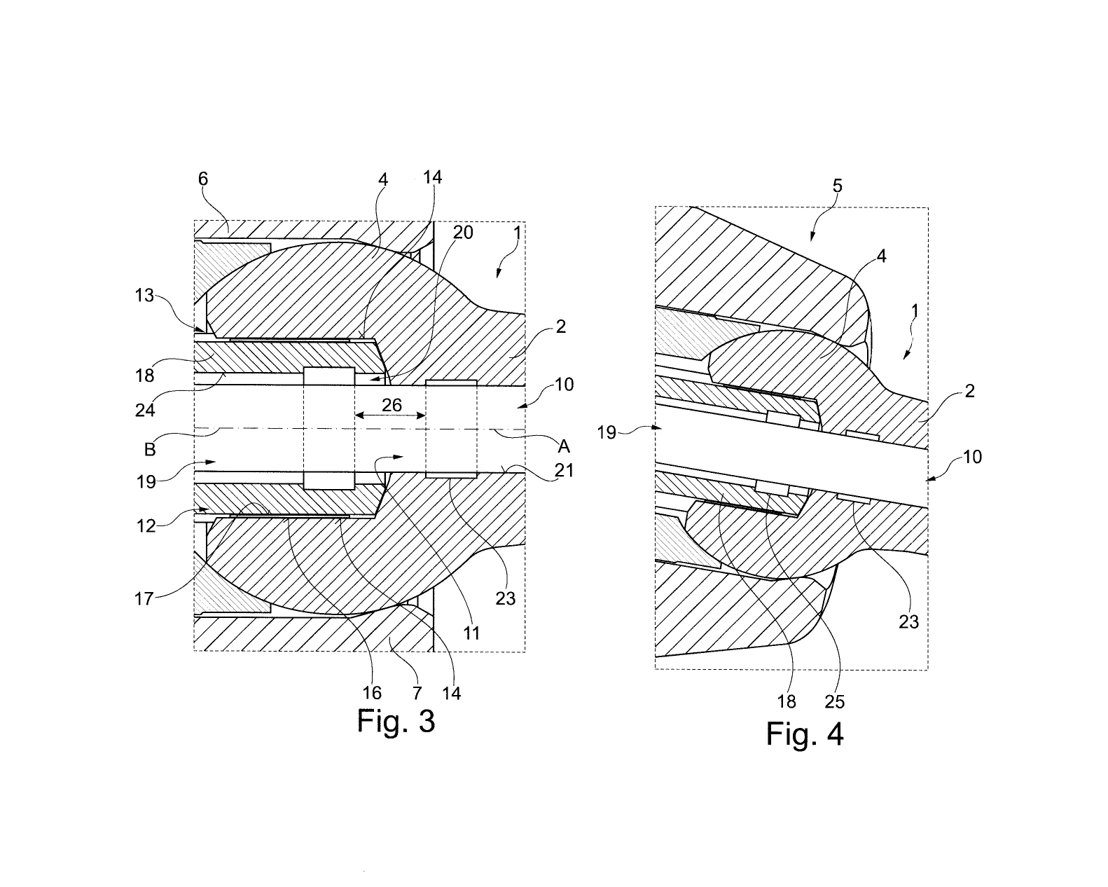

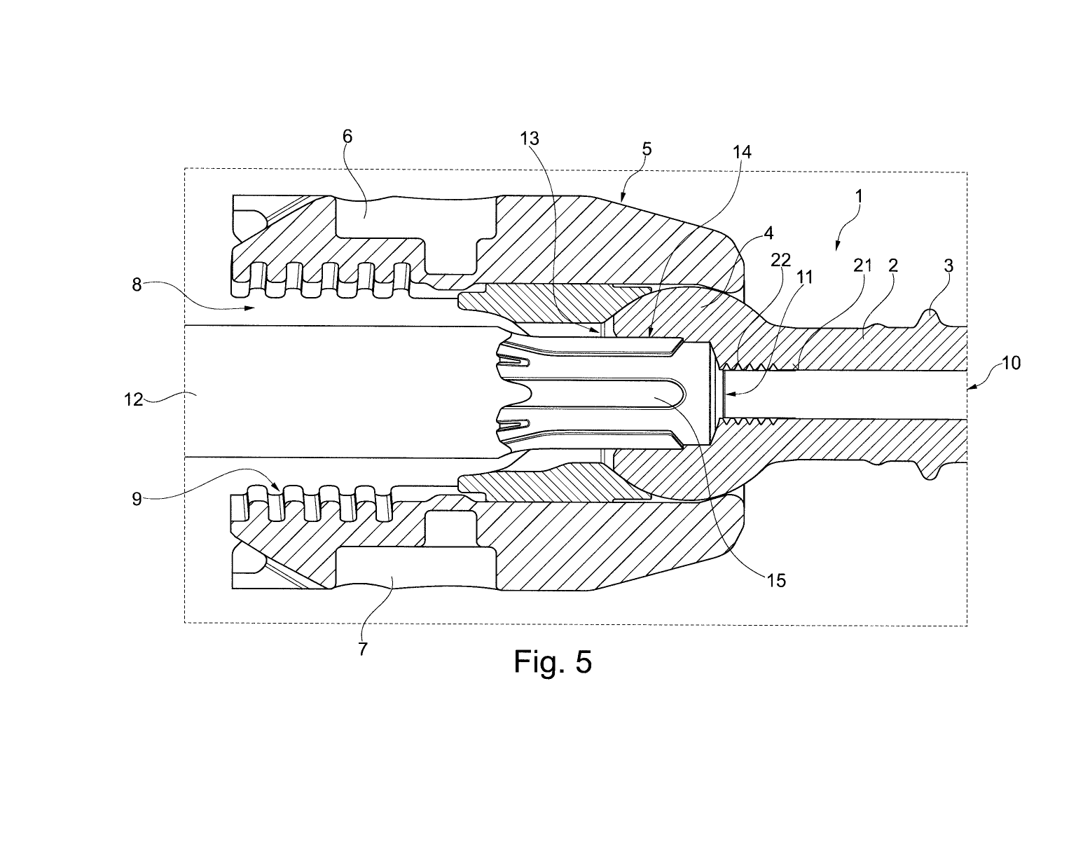

The ball-shaped screw head 4 comprises a central tool receiver 13 in the form of a recess. As can be clearly seen in particular in FIG. 5, the tool receiver 13 comprises an engagement contour 14 designed as a Torx socket for a correspondingly shaped tool contour 15 of the implantation instrument 12. Furthermore, as shown in FIGS. 1 and 3, the tool receiver 13 has formed therein a connection contour 16, here in the form of a female thread 16, which superimposes with the engagement contour 14. The female thread 16 serves for a screwing engagement with a connection structure 17 of the implantation instrument 12, here in the form of a male thread 17 with mating design.

FIGS. 1 and 3 show that the implantation instrument 12 comprises an injection cannula 18 having a longitudinal axis B. In said injection cannula, an inner channel 19 is formed along the longitudinal axis B, which at the distal end of the injection cannula 18 has a distal outlet opening 20. The inner channel's 19 opposite end, which is not illustrated in the Figures, is fluidically connected to a reservoir or container for bone connection material. The inner channel 19 serves to convey bone connection material from said reservoir to the outlet opening 20, and from the latter via the proximal inlet opening 11 into the injection channel 10 of the pedicle screw 1.

To this end, the implantation instrument 12 can be coupled to the pedicle screw 1 in the manner described in the following: Via the female thread 16 and the male thread 17, the implantation tool 12 is placed and retained in the tool receiver 13 of the screw head 4 such that the inner channel 19 and the injection channel 10 form a flow path from the cement reservoir to the (not shown) outlet openings of the pedicle screw 1, which flow path is tight towards outside for the bone connection material. With such an intended arrangement of the instrument 12 in the tool receiver 13, also the engagement contour 14 of the pedicle screw 1 engages the tool contour 15 of the implantation instrument 12 in such a manner that the pedicle screw 1 can be screwed into a bone with the implantation instrument 12. When the intended end position of the pedicle screw 1 is reached, bone connection material is introduced via the injection cannula. Said material flows through the inner channel 19 and the injection channel 10 to the outlet openings of the pedicle screw 1 and out of these into cavities present between the pedicle screw 1 and the bone substance. After a certain period of time, the material is at least partially hardened and solidified so that the implantation instrument 12 can be detached from the pedicle screw 1 and removed therefrom.

To this end, the female thread 16 and the male thread 17 are simply unscrewed, whereby the instrument 12 and its injection cannula 18 are removed from the screw head in axial direction. In this process, the inventive design of the channels 10, 19 comprising an offset comes into effect. With the embodiment of FIGS. 1 and 2, a female thread 22 is machined in the injection channel 10 in the wall 21 in an area adjoining the proximal inlet opening 11, said female thread forming an offset or several offsets. In the embodiment of FIGS. 3 and 4, an undercut 23 is machined in the wall 21 of the injection channel 10. FIGS. 1 to 4 show that the wall 24 of the inner channel 19 of the injection cannula 18 has an offset as well, here in each case in the form of an undercut 25. An alternative is represented by a female thread which is not illustrated in the Figures.

Any bone connection material which is present in the channels 10, 19, see for instance in FIG. 5, penetrates into the screw threads of the female thread 22 or into the undercuts 23, 25 in the course of the injection process and hardens there. It could be said that the offset of the channels 10, 19 in the form of the female thread 22 or the undercuts 23, 25 is casted in the bone connection material, the latter hardening in said shape. Due to the positive fit with the corresponding offset, the material cannot be positioned in the direction of the respective longitudinal axis A or B. Upon removing the implantation instrument 12 in axial direction away from the pedicle screw 1, the bone connection material which is present within the channels 10, 19 will break or shear off in a (predetermined breaking) area 26 situated between the two undercuts 23 and 25 or between the undercut 25 and the female thread. This ensures that hardened bone connection material breaks off as close as possible to the proximal inlet opening 11 of the channel 10 in the pedicle screw 1 or the outlet opening 20 of the channel 19 in the injection cannula 18 and does not protrude too far.

In above embodiment, the injection cannula 18 is part of the implantation tool 12. The injection cannula 18 may also be a separate component that can be screwed into the pedicle screw 1 and likewise is provided with an undercut 25 or radial offset.

1. A bone implant comprising a distal implant shank formed for being embedded in a bone, and a proximal implant head having a tool receiver for an implantation tool, from which an injection channel for injecting a bone connection material extends into the implant shank and forms a predetermined injection channel diameter,
wherein the injection channel comprises a proximal inlet opening and a wall surrounding the injection channel, and wherein the wall, starting from the tool receiver, comprises a radial offset machined in the wall of the injection channel, the radial offset extending radially outwards with respect to the predetermined injection channel diameter proximally and distally relative to the radial offset.

2. The bone implant according to claim 1, wherein the radial offset is formed by at least one step or shoulder machined in the wall of the injection channel and/or is formed so as to circumferentially extend in a circumferential direction at least in sections.

3. The bone implant according to claim 1, wherein the radial offset is formed by an undercut machined in the wall of the injection channel.

4. The bone implant according to claim 1, wherein the radial offset is formed in an area of the wall which is spaced from the proximal inlet opening of the injection channel at least by about 0.5 mm and at most about 10 mm.

5. The bone implant according to claim 1, wherein the radial offset in a radial direction amounts to between about 0.1 mm to 3 mm.

6. The bone implant according to claim 1, wherein the bone implant is a bone screw, the implant head is formed as a ball head, and the tool receiver is configured for receiving a screw-driving tool, wherein the proximal inlet opening is formed in a distal boundary surface of the tool receiver.

7. The bone implant according to claim 1, wherein the tool receiver comprises a connection structure for a detachable connection with an injection cannula for injection of bone connection material, and/or the tool receiver comprises an engagement contour for an implantation tool by which the bone implant is implanted during an implantation.

8. An implantation instrument for a bone implant according to claim 1, the implantation instrument comprising an injection cannula including an inner channel having a longitudinal axis for injection of bone connection material and a second wall surrounding the inner channel, wherein the inner channel comprises a distal outlet opening for bone connection material and has a predetermined inner channel diameter, wherein the second wall of the injection cannula has a second radial offset with respect to the longitudinal axis, the second radial offset starting from the distal outlet opening and being machined in the second wall of the inner channel, the second radial offset extending radially outwards with respect to the predetermined inner channel diameter proximally and distally relative to the second radial offset.

9. The implantation instrument according to claim 8, wherein the second radial offset of the injection cannula is formed by at least one step or shoulder machined in the second wall of the inner channel and/or is formed so as to circumferentially extend in the circumferential direction at least in sections.

10. The implantation instrument according to claim 8, wherein the second radial offset of the injection cannula is formed by a second undercut machined in the second wall of the inner channel.

11. The implantation instrument according to claim 8, wherein the second radial offset of the injection cannula is formed in an area of the second wall which is spaced from the distal outlet opening at least by about 0.5 mm and at most about 10 mm.

12. The implantation instrument according to claim 8, wherein the injection cannula comprises a connection structure for a detachable connection with the tool receiver of the bone implant.

13. The implantation instrument according to claim 8, wherein the injection cannula is designed as an implantation tool and comprises a tool contour matched with the tool receiver.

14. An implantation system comprising:
a bone implant comprising a distal implant shank formed for being embedded in a bone, and a proximal implant head having a tool receiver for an implantation tool, from which an injection channel for injecting a bone connection material extends into the implant shank and forms a predetermined injection channel diameter, wherein the injection channel comprises a proximal inlet opening and a wall surrounding the injection channel, and wherein the wall, starting from the tool receiver, comprises a radial offset machined in the wall of the injection channel, the radial offset extending radially outwards with respect to the predetermined injection channel diameter proximally and distally relative to the radial offset; and
an implantation instrument comprising a injection cannula including an inner channel having a longitudinal axis for injection of bone connection material and a second wall surrounding the inner channel, wherein the inner channel comprises a distal outlet opening for bone connection material and has a predetermined inner channel diameter, wherein the second wall of the injection cannula has a second radial offset with respect to the longitudinal axis, the second radial offset starting from the distal outlet opening and being machined in the second wall of the inner channel, the second radial offset extending radially outwards with respect to the predetermined inner channel diameter proximally and distally relative to the second radial offset.
